(19) United States
(12) Patent Application Publication
(10) Pub. No.: US 2022/0346353 A1
(43) Pub. Date: Nov. 3, 2022

(54) Canine Urine Collection Apparatus

(71) Applicant: Michele Alexander, Hillsboro, OR (US)

(72) Inventor: Michele Alexander, Hillsboro, OR (US)

(21) Appl. No.: 17/243,827

(22) Filed: Apr. 29, 2021

(51) Int. Cl. A01K 23/00; A61D 99/00

(52) U.S. Cl. CPC A01K 23/00; A61D 99/00; A61B 10/007

(57) ABSTRACT

A canine urine collection apparatus for taking a sample of canine urine includes a garment body having a body inner side and a body outer side. The garment body has a bag aperture extending from the body inner side through the body outer side. The garment body is worn around a torso of a canine with the bag aperture aligned with a urethra of the canine. The plurality of engagement members is coupled to the garment body. The plurality of engagement members selectively secures and releases the garment body to the torso of the canine. A collection bag is coupled to the garment body. The collection bag has a bag perimeter selectively engaged to the garment body around the bag aperture. The collection bag collects a urine sample from the canine.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to urine collection devices and more particularly pertains to a new urine collection device for taking a sample of canine urine. The present device includes a wearable garment secured around the torso of the canine. There is a detachable collection bag coupled to the garment.

### (2) Description of Related Art Including Information Disclosed Under 37 CFR 1.97 and 1.98

The prior art relates to urine collection devices. Known devices include a pan or collection funnel on a stick. Few devices utilize a wearable garment to secure a collection bag in place. These devices include straps coupled to the collection bag rather than having a selectively removable bag coupled to a secured garment.

### BRIEF SUMMARY OF THE INVENTION

An embodiment of the disclosure meets the needs presented above by generally comprising a garment body having a body inner side and a body outer side. The garment body has a bag aperture extending from the body inner side through the body outer side. The garment body is configured to be worn around a torso of a canine with the bag aperture aligned with a urethra of the canine. The plurality of engagement members is coupled to the garment body. The plurality of engagement members selectively secures and releases the garment body to the torso of the canine. A collection bag is coupled to the garment body. The collection bag has a bag perimeter selectively engaged to the garment body around the bag aperture. The collection bag is configured to collect a urine sample from the canine.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a canine urine collection apparatus according to an embodiment of the disclosure.

FIG. 2 is a bottom plan view of an embodiment of the disclosure.

FIG. 3 is a top plan view of an embodiment of the disclosure.

FIG. 4 is a side elevation view of an embodiment of the disclosure.

FIG. 5 is an in-use view of an embodiment of the disclosure.

FIG. 6 is an isometric view of an embodiment of the disclosure.

FIG. 7 is a bottom plan view of an embodiment of the disclosure.

FIG. 8 is an in-use view of an embodiment of the disclosure.

FIG. 9 is a bottom plan view of an embodiment of the disclosure.

FIG. 10 is an in-use view of an embodiment of the disclosure.

FIG. 11 is an in-use detail view of an embodiment of the disclosure.

FIG. 12 is a flow chart view of an embodiment of the disclosure.

### DETAILED DESCRIPTION OF THE INVENTION

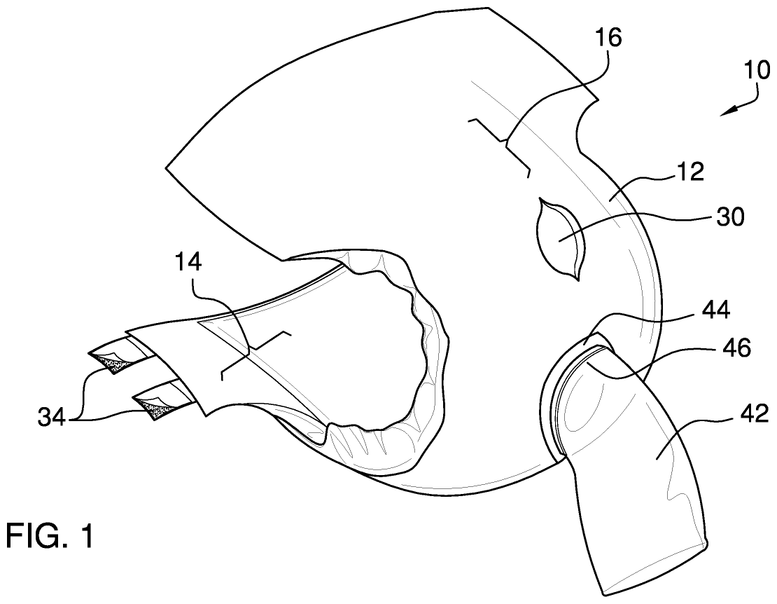

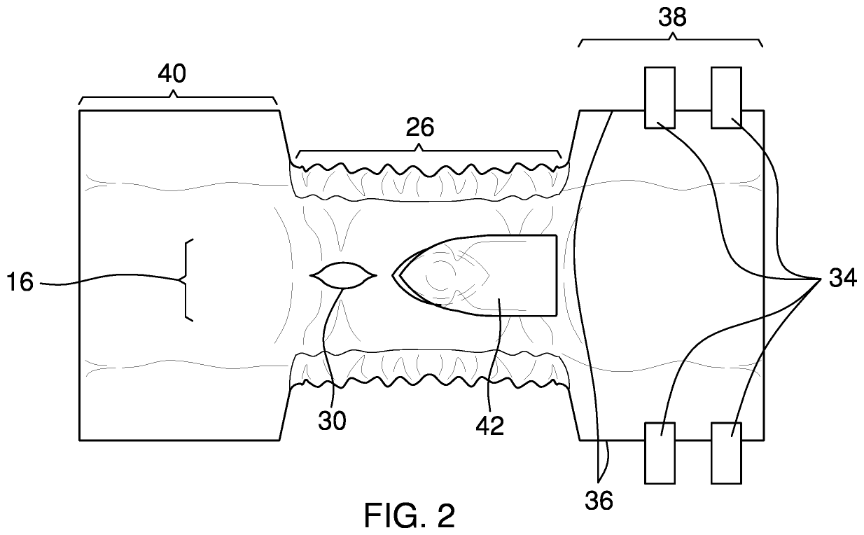

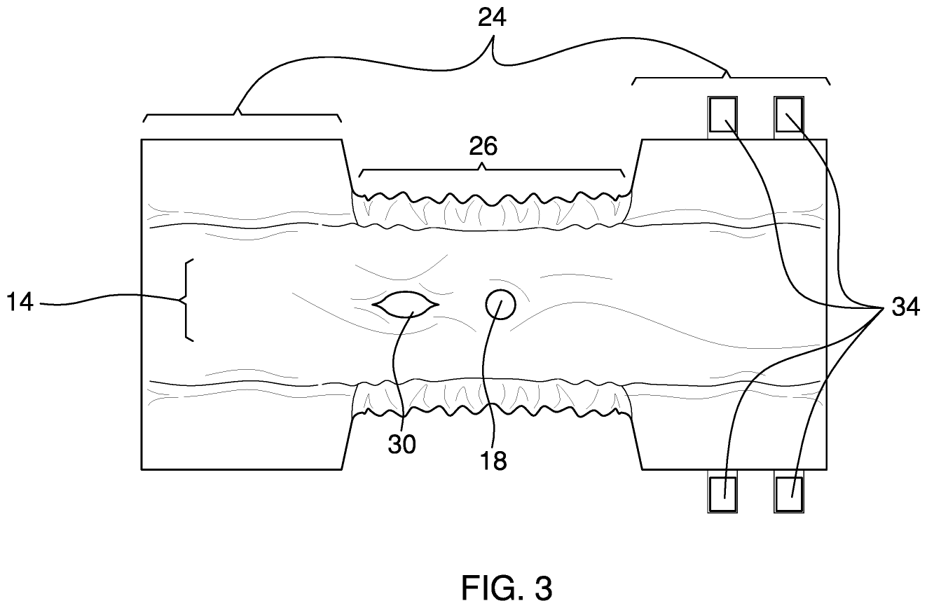

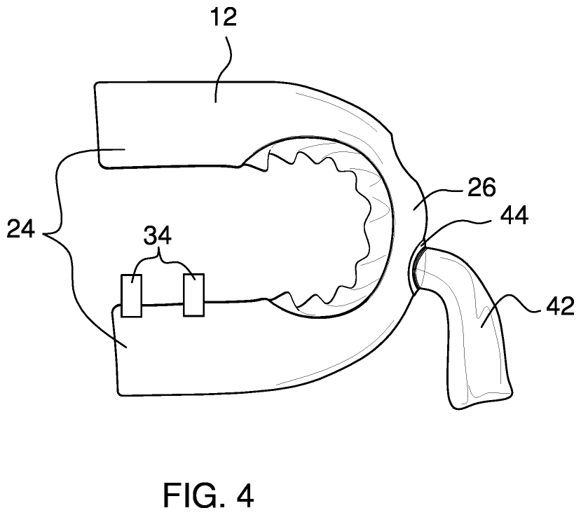

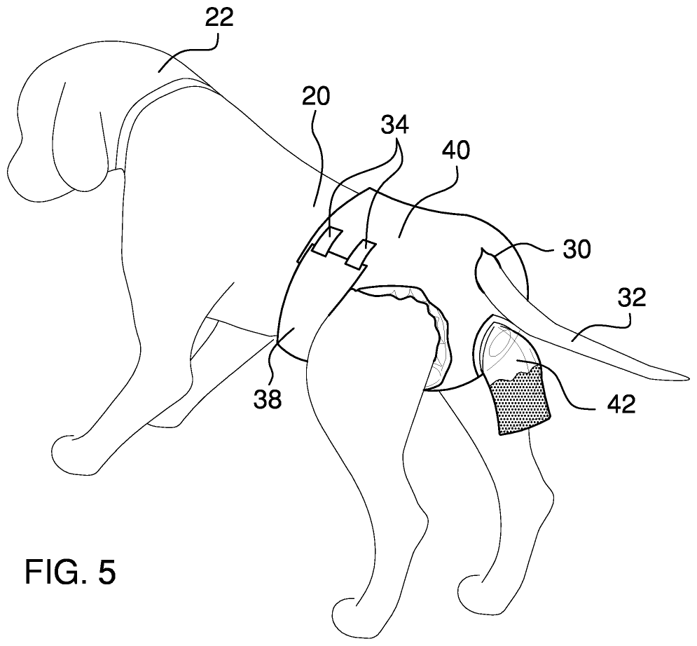

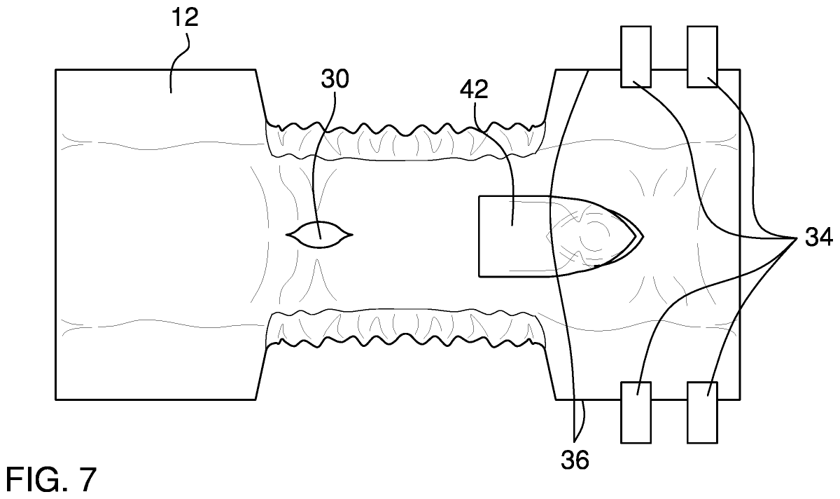

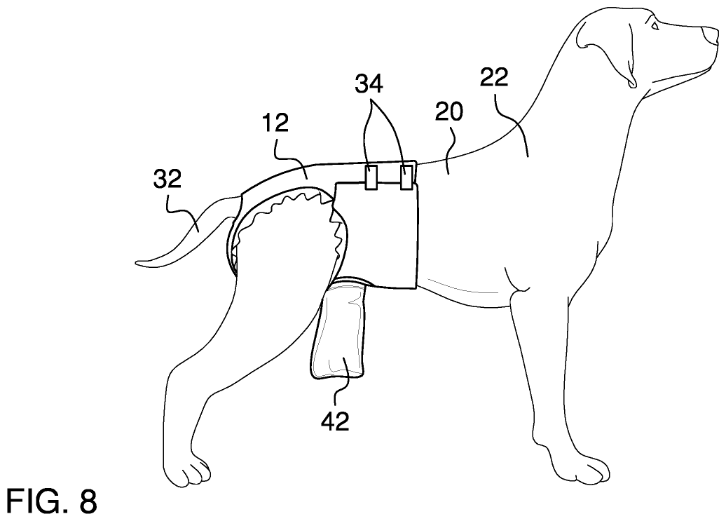

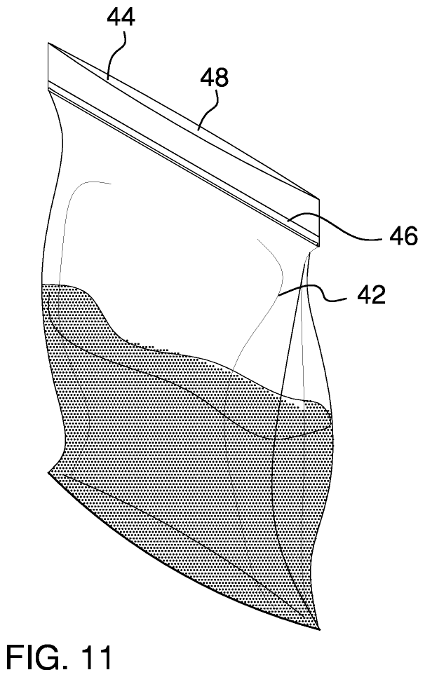

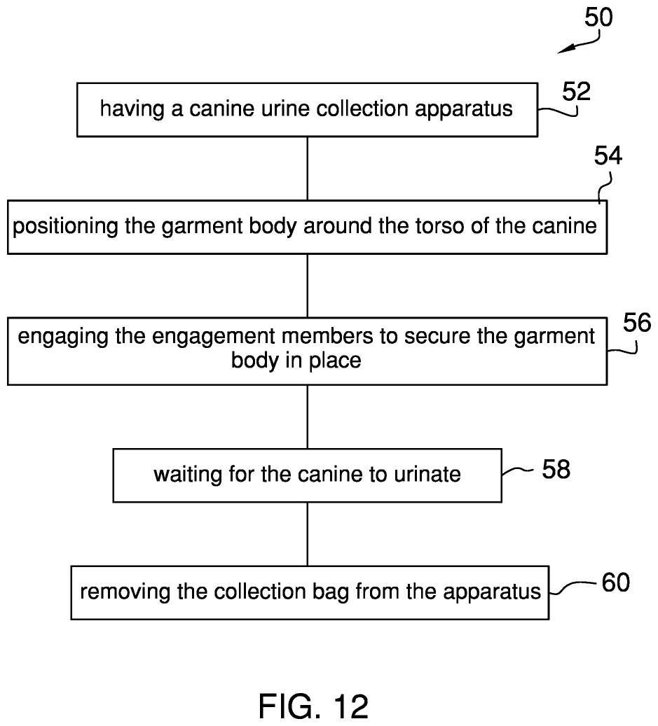

With reference now to the drawings, and in particular to FIGS. 1 through 12 thereof, a new urine collection device embodying the principles and concepts of an embodiment of the disclosure and generally designated by the reference numeral 10 will be described.

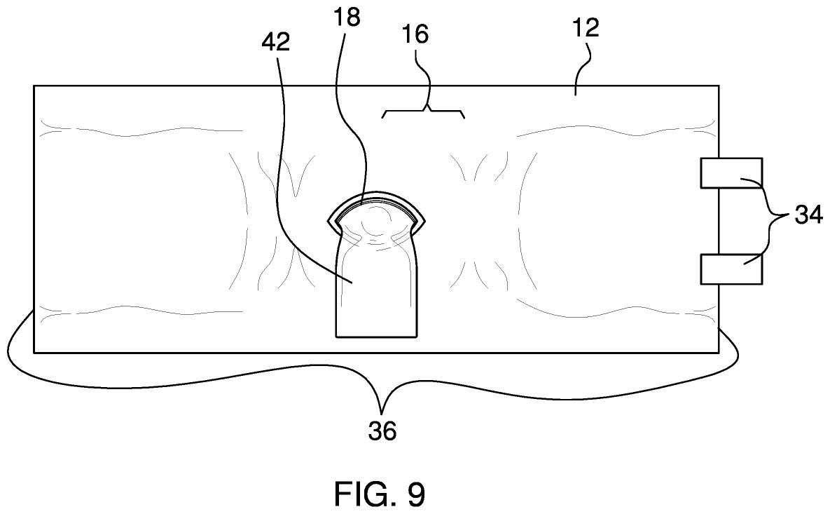

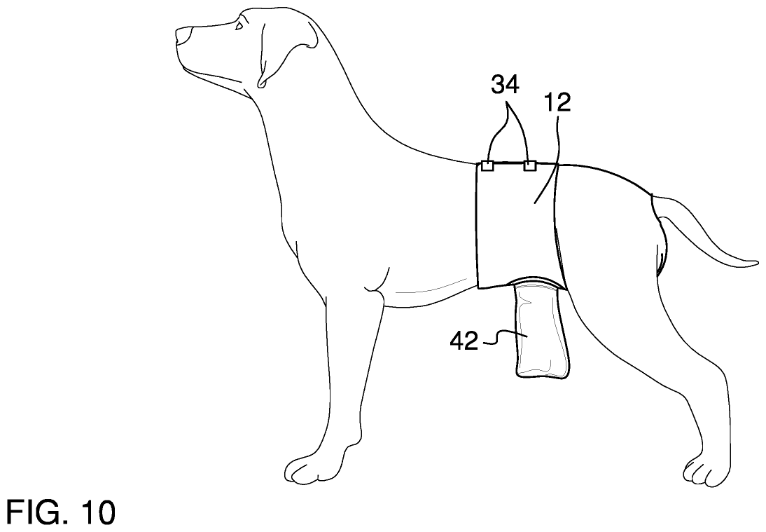

As best illustrated in FIGS. 1 through 12, the canine urine collection apparatus 10 generally comprises a garment body 12 having a body inner side 14 and a body outer side 16. The garment body 12 has a bag aperture 18 extending from the body inner side 14 through the body outer side 16. The garment body 12 is configured to be worn around a torso 20 of a canine 22 with the bag aperture 18 aligned with a urethra of the canine. The garment body 12 may be rectangular for male canines as shown in FIGS. 9 and 10. The body inner side 14 may be absorptive and the garment body 12 may generally be a diaper-like material.

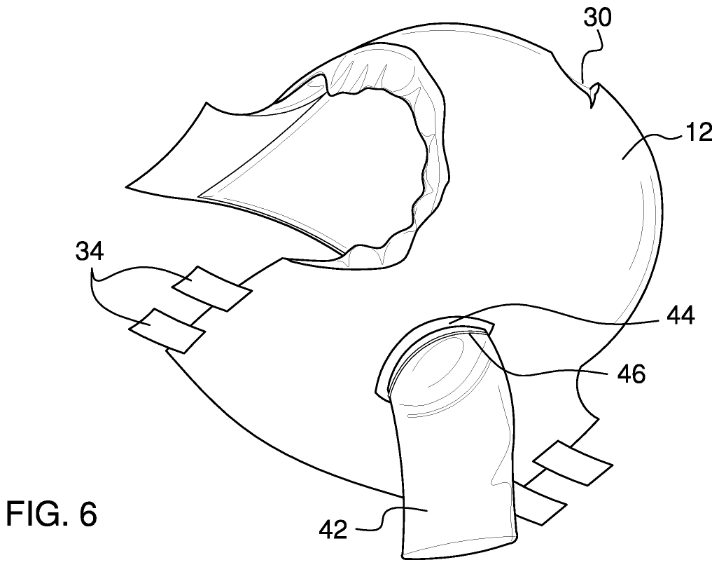

The garment body 12 may alternatively have a pair of outer portions 24 and a medial portion 26. The pair of outer portions 24 is coupled around the torso 20 of the canine and the medial portion 26 extending around a rear end 28 of the canine. The medial portion 26 has a tail aperture 30 extending from the body inner side 14 through the body outer side 16 and configured to receive a tail 32 of the canine. The bag aperture 18 also extends through the medial portion 26 and is positioned appropriately for female dogs (shown in FIGS. 1-5) or for male dogs (shown in FIG. 6-8). The medial portion 26 may be elasticized and may be thinner than the pair of outer portions 24.

A plurality of engagement members 34 is coupled to the garment body 12. The plurality of engagement members 34 selectively secures and releases the garment body 12 to the torso 20 of the canine. The plurality of engagement members 34 may extend from a pair of body side edges 36 of the garment body. The plurality of engagement members 34 may be coupled to a first outer portion 38 of the pair of outer portions and selectively engages a second outer portion 40 of the pair of outer portions. The plurality of engagement members 34 may be selectively engageable fasteners such as, but not limited to, adhesives, hook-and-loop, snaps, and the like.

A collection bag 42 is coupled to the garment body 12. The collection bag 42 has a bag perimeter 44 selectively engaged to the garment body 12 around the bag aperture 18. The collection bag 42 may have a closure strip 46 to selectively seal and unseal the collection bag 42. A bag inside 48 of the collection bag may be adhesive between the closure strip 46 and the bag perimeter 44 to adhere to the body outer side 16 around the bag aperture 18. The collection bag 42 is configured to collect a urine sample from the canine 22.

In use, the canine urine collection apparatus 10 allows for a method of use 50 comprising a step 52 comprising having the canine urine collection apparatus 10. A step 54 comprises positioning the garment body 12 around the torso 120 of the canine. A step 56 comprises engaging the engagement members 34 to secure the garment body 12 in place. A step 58 comprises waiting for the canine 18 to urinate. A step 60 comprises removing the collection bag 42 from the apparatus 10.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A canine urine collection apparatus comprising:
a garment body having a body inner side and a body outer side, the garment body having a bag aperture extending from the body inner side through the body outer side, the garment body being configured to be worn around a torso of a canine with the bag aperture aligned with a urethra of the canine;
a plurality of engagement members coupled to the garment body, the plurality of engagement members selectively securing and releasing the garment body to the torso of the canine; and
a collection bag coupled to the garment body, the collection bag having a bag perimeter selectively engaged to the garment body around the bag aperture, the collection bag being configured to collect a urine sample from the canine.

2. The canine urine collection apparatus of claim 1 further comprising the collection bag having a closure strip, the closure strip selectively sealing and unsealing the collection bag.

3. The canine urine collection apparatus of claim 2 further comprising a bag inside of the collection bag being adhesive between the closure strip and the bag perimeter.

4. The canine urine collection apparatus of claim 1 further comprising the garment body having a pair of outer portions and a medial portion, the pair of outer portions being coupled around the torso of the canine and the medial portion extending around the rear end of the canine, the medial portion having a tail aperture extending from the body inner side through the body outer side and configured to receive a tail of the canine.

5. The canine urine collection apparatus of claim 4 further comprising the plurality of engagement members being coupled to a first outer portion of the pair of outer portions and selectively engaging a second outer portion of the pair of outer portions.

6. The canine urine collection apparatus of claim 5 further comprising the plurality of engagement members extending from a pair of body side edges of the garment body.

7. The canine urine collection apparatus of claim 4 further comprising the medial portion being elasticized.

8. The canine urine collection apparatus of claim 1 further comprising the body inner side being absorptive.

9. A canine urine collection apparatus comprising:
a garment body having a body inner side and a body outer side, the garment body having a bag aperture extending from the body inner side through the body outer side, the garment body being configured to be worn around a torso of a canine with the bag aperture aligned with a urethra of the canine, the garment body having a pair of outer portions and a medial portion, the pair of outer portions being coupled around the torso of the canine and the medial portion extending around the rear end of the canine, the medial portion being elasticized, the medial portion having a tail aperture extending from the body inner side through the body outer side and configured to receive a tail of the canine, the body inner side being absorptive;
a plurality of engagement members coupled to the garment body, the plurality of engagement members selectively securing and releasing the garment body to the torso of the canine, the plurality of engagement members extending from a pair of body side edges of the garment body, the plurality of engagement members being coupled to a first outer portion of the pair of outer portions and selectively engaging a second outer portion of the pair of outer portions; and
a collection bag coupled to the garment body, the collection bag having a bag perimeter selectively engaged to the garment body around the bag aperture, the collection bag having a closure strip, the closure strip selectively sealing and unsealing the collection bag, a bag inside of the collection bag being adhesive between the closure strip and the bag perimeter, the collection bag being configured to collect a urine sample from the canine.

10. A method of collecting a canine urine sample comprising:
having a canine urine collection apparatus comprising:
a garment body having a body inner side and a body outer side, the garment body having a bag aperture extending from the body inner side through the body outer side, the garment body being configured to be worn around a torso of a canine with the bag aperture aligned with a urethra of the canine;
a plurality of engagement members coupled to the garment body, the plurality of engagement members selectively securing and releasing the garment body to the torso of the canine; and
a collection bag coupled to the garment body, the collection bag having a bag perimeter selectively engaged to the garment body around the bag aperture, the collection bag being configured to collect a urine sample from the canine;
positioning the garment body around the torso of the canine;
engaging the engagement members to secure the garment body in place;
waiting for the canine to urinate; and
removing the collection bag from the apparatus.
